(19) United States
(12) Patent Application Publication
(10) Pub. No.: US 2010/0184969 A1
(43) Pub. Date: Jul. 22, 2010

(54) Process to Prepare Sucralose

(76) Inventors: Mangesh Rajadhyaksha, Mumbai (IN); Ajit Shirwaiker, Mumbai (IN)

Correspondence Address: LADAS & PARRY LLP, 26 WEST 61ST STREET, NEW YORK, NY, 10023, US

(21) Appl. No.: 12/084,214

(22) PCT Filed: Nov. 7, 2006
(86) PCT No.: PCT/IN2006/000461

§ 371 Date: Apr. 28, 2008

(30) Foreign Application Priority Data
Nov. 8, 2005 (IN) 1387/MUM/2005

(51) Int. Cl. C07H 5/02

(52) U.S. Cl. 536/122

(57) ABSTRACT

The present invention relates to a process to prepare sucralose. More particularly the said process relates to the production of sucralose, chemically known as, 6-Dichloro-1,6-dideoxy-β-D-fructofuranosyl-4-chloro-4-deoxy-alpha-D-galactopyranoside of formula (1)

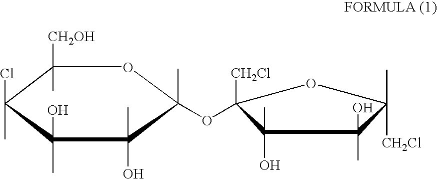

The present invention relates to a process to prepare sucralose. More particularly the said process relates to the production of sucralose, chemically known as 1,6-Dichloro-1,6-dideoxy-β-D-fructofuranosyl-4-chloro-4-deoxy-alpha-D-galactopyranoside of formula (1) herein below:

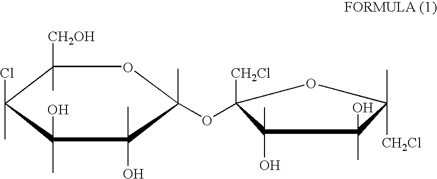

### BACKGROUND OF THE INVENTION

Sucralose is a low calorie sweetener made from sugar and tastes similar to sugar. It is about 600 times sweeter than sugar. Sucralose can be safely consumed and used wherein there is a need to avoid use of sugar. More particularly it is very useful for preparing food, beverages and nutritional product wherein the use of sugar needs to be avoided. The sucralose is used in foods sweetening beverage and nutritional products ingredient worldwide.

Sucralose is also important, from the point of view of calorie cautious people. The products sweetened with sucralose can help consumers reduce/eliminate the addition of sugar and thereby reduce the calories in the diet. Sucralose can be safely consumed as it has been conclusively proved by more than 100 scientific studies conducted over a 20-year period that sucralose is safe for consumption. The Joint FAO/WHO Expert Committee on Food Additives (JECFA) was the first regulatory body to endorse the safety of sucralose in 1990.

Canada's Health Protection Branch became the first national regulatory agency to endorse sucralose safety and permit its use in foods and beverages in 1991. In 1998, the United States Food and Drug Administration (FDA) approved the use of sucralose in 15 food and beverage categories—the broadest initial approval ever given to a food additive which was further extended in August 1999 to cover use as a general purpose sweetener in all foods, beverages, dietary supplements and medical foods. European Union also permitted the use of sucralose as sweetener in a broad range of food and beverage products. Sucralose is now permitted for use in over 60 countries and has been consumed by millions of people worldwide. International experts in the field of different scientific disciplines, including toxicology, oncology, teratology, neurology, hematology, pediatrics and nutrition and the studies with highest scientific standards, have clearly demonstrated that sucralose is not carcinogenic.

The categories of product wherein sucralose can be used as a low calorie sweetener including food, soft drinks, ice creams, bakery items, dairy products, variety of sweets especially for diabetics, applications requiring heat treatment, requiring long storage, beverages, alcoholic beverages and beverage mixes; flavorings, jams and jellies, meat products, milk products, processed fruits and fruit juices, processed vegetables and vegetable juices, snack foods, soft candy, soups and soup mixes, sugar substitutes, sweet sauces, toppings and many many other products.

Sucralose belongs to a category of sweeteners called “non-nutritive,” meaning that they do not provide a significant number of calories. Saccharin, aspartame and acesulfame-k are other examples of non-nutritive sweeteners.

### PRIOR ART

Conventionally sucralose is prepared in multiple-step manufacturing process that substitutes 3 chlorine atoms for the hydroxyl groups on a sugar molecule. The tight molecular bond between the chlorine atoms and the rest of the sugar molecule results in a very stable molecule making it difficult for the metabolism in the body for calories.

The known prior art process for the preparation of sucralose comprise converting sucrose to tri trityl sucrose by tritylation, acetylating the remaining hydroxy groups in tri trytil sucrose using acetic anhydride to obtain tri trytil pentaacetate (TRISPA), detritylateing TRISPA to 2,3,4,3′,4′ penta-o-acetyl sucrose (4-PAS), isomerising 4-PAS to 2,3,6,3′,4′ penta-o-acetyl sucrose (6-PAS), replacing three hydroxyl groups in 6-PAS by chlorine by chlorination, preferably with thionyl chloride to obtain 4,1′,6′-trichloro-4,1′,6-tridoxygalactosucrose (TOSPA), deacetylating TOSPA using sodium methoxide to obtain sucralose.

U.S. Pat. No. 4,826,962 (Rathbone et al. May 2, 1989) describes an enzymatic process for the preparation of sucralose comprising incubating novel chlorinated sugar O-α-D-6-chloro-6-deoxygalactopyranosyl-(1→6)-α-D-4-chloro-4-deoxygalactopyranosyl-(1→2)-β-D-1,6-dichloro-1,6-dideoxy-fructofuranoside (TCR) in the presence of an enzyme serving to remove the β-chloro-β-deoxygalactosyl moiety from the 6-position. The enzyme therein was especially derived from a strain of Mortierella vinacea, Circinella muscae or Aspergillus niger.

U.S. Pat. No. 5,498,709 (Navia Mar. 12, 1996, et al.) provides for preparation of sucralose from sucralose-6-ester whereby the sucralose-6-ester is deacylated directly either prior to or after removal of the tertiary amide reaction vehicle from the neutralized chlorination reaction mixture, to produce an aqueous solution of sucralose plus salts and impurities, from which sucralose is recovered by extraction and is then preferably purified by crystallization.

Another U.S. Pat. No. 4,920,207 (Sankey et al.) provides for selective removal of trityl groups from TRISPA which comprises adding a catalytic amount of an aralkyl chloride or hydrogen chloride to a solution of TRISPA in an inert organic solvent and hydrogenating in the presence of a hydrogenolysis catalyst such a platinum or palladium. However these are industrially not viable because of use of costly platinum or palladium catalyst.

The prior art process of converting TRISPA to 4-PAS involves use of toluene-HCL or methanolic-HCL or platinum or palladium-HCL and alkyl chloride reagents. It has been observed that the use of these reagents do not give products facilitating the manufacture on industrial scale. Conventionally, the process isolates the producut 4-PAS before proceeding to the next step of isomerisation of its 6-PAS whereas process in the present invention the said process is done in-situ.

It is observed that if the conventional solvent system consisting of toluene, methylene chloride, methanol and HCl gas is replaced with the system consisting of Chloroform and HCL gas, the reaction is clean and minimum impurities are formed which helps in facilitating the production of sucralose on industrial scale.

The conventional reagent for conversion of TOSPA to sucralose consists of alkaline earth metal hydroxides such as sodium methoxide, barium hydroxide in methanol and further acidification is done by ion exchange resins. This probably increases the ash content in sucralose when ethyl acetate is used as a solvent during the process workup, thereby causing difficulties in the product passing the Food Chemical Codex (FCC) limits. The inventors of the present invention have observed that if this reagent is replaced by tetraalkyl ammonium hydroxide such as tetramethyl or tetrabutyl ammonium hydroxide in methanol and the acidification done by methanolic HCl, the ash content in sucralose produced considerably reduces, remaining within the FCC limits.

Further the ion exchanger resins used for acidification are comparatively costlier than methanolic HCl thereby giving economic advantage over the known prior art.

Therefore, the primary object of the present invention is to provide a process for the production of sucralose which uses different reaction mixtures giving products which can be used conveniently and without problem for further down steam processing in production of sucralose.

Another object is to provide a process wherein the production of 4-PAS is in situ thereby eliminating product isolation and making it easier to handle and reducing difficulties in manufacture of sucralose.

A better and more complete understanding of the invention may be garnered from the detailed description of the preferred embodiment of the best mode of the invention as contemplated by the inventor

### DESCRIPTION OF THE INVENTION

Accordingly the present invention provides a process for the preparation of sucralose of formula (1)

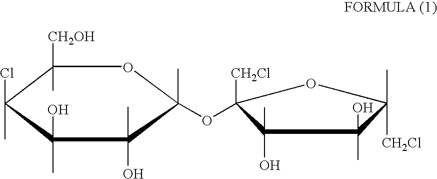

Comprises
a) Preparing 6,1,6′-tri-o-trityl sucrose pentaacetate (TRISPA) of formula (2) from sucrose using triethyl amine or pyridine as a base

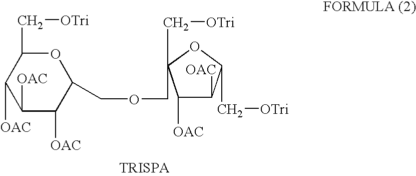

b) preparing 2,3,6,3′,4′-Penta-o-acetylsucrose (6-PAS) of FORMULA (4) using TRISPA of formula (2) via in-situ preparation of compound 4-PAS of formula (3)

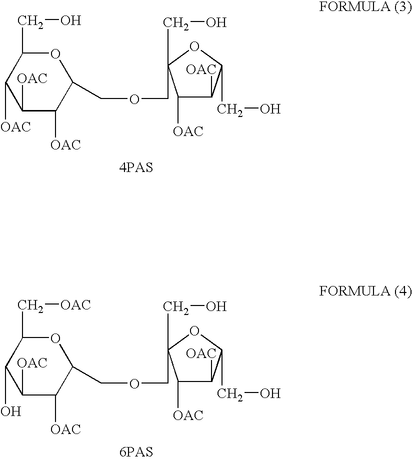

c) Subjecting 6-PAS to chlorination to obtain compound TOSPA of formula (5) by using aqueous methanol during workup

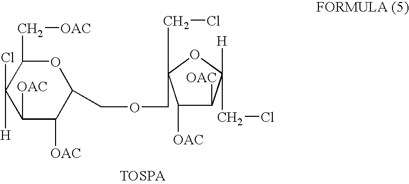

d) De-acetylating TOSPA for formula (5) to sucralose of formula (1) using quaternary ammonium hydroxide.

In one of the embodiments of the present invention the process for the preparation of sucralose comprises,
a) preparing a mixture of sucrose, triethyl amine and dimethyl amino pyridine as a catalyst in N-N-dimethyl formamide (DMF) at ambient temperature, adding excess trityl chloride successively in three equal portions in 1 to 1.5 hrs, continuing stirring at ambient temperature for about 16 hrs., distilling DMF under vacuum below 85° C., to obtain a thick paste of crude tritrytil sucrose, dissolving the thick paste in acetic anhydride at a temperature in the range of 50-60° C., adding anhydrous sodium acetate while maintaining the reaction temperature at 110-115° C. for 3 to 4 hrs., cooling the reaction mixture to 45-50° C., precipitating by adding methanol, separating and washing the solid successively with methanol and water to remove inorganic salts, drying the solid at 65-75° C. till constant weight to obtain TRISPA; or alternatively
b) preparing a mixture of sucrose in pyridine, adding to this mixture excess trityl chloride successively in three equal portions in 1 hrs, at ambient temperature, heating the reaction mixture to 45 to 50° C. for a minimum of 5 hrs. distilling pyridine under vacuum at a temperature below 80-85° C. to obtain the residue, cooling the residue to 50° C., adding acetic anhydride to obtain a homogenous mass, stirring the homogenous mass at ambient temperature (75° C. to 80° C.) for 3 to 3.5 hrs. heating the homogenous mass to 100-105° C. for minimum two hours, precipitating by adding methanol, separating and washing the solid successively with methanol and water to remove inorganic salts, drying the solid at 65-75° C. till constant weight to obtain TRISPA;
c) dissolving dry TRISPA in chloroform, cooling the clear solution to a minimum of −1° C. to −15° C., passing dry HCL gas through the solution for a maximum of about 15 to 60 mins., to obtain a reaction mixture having temperature of −5 to −3° C., quickly pouring the reaction mixture into aqueous Sodium bicarbonate solution under constant stirring at pH 7.5 to 8 separating the chloroform layer containing 4-PAS and trytinol, separating the aqueous layer, washing the aqueous layer 2 to 3 times with chloroform, removing the chloroform by distillation to obtain the thick pasty mass of a mixture containing 4-PAS and tritinol, extracting the thick pasty mass with hot water of 85-90° C., 3 to 4 times to obtain 4-PAS in aqueous solution, cooling the hot water extract to 25-30° C., saturating with sodium chloride, extracting the saturated solution with methylene chloride 3-4 times, distilling the methylene chloride layer to obtain the semi to semi solid 4-PAS; or alternatively
d) dissolving wet or dry TRISPA in a toluene, removing water by conventional methods if wet TRISPA is used, cooling the clear solution to −1 to −15° C., passing dry HCL gas through the clear solution for a maximum of 15 to 60 mins., to obtain a reaction mixture having temperature of −5 to −3° C., quickly adding aqueous sodium bicarbonate into the reaction mixture under constant stirring at pH 7.5 to 8 separating the toluene layer containing mixture of trytil chloride(approx 85-90%) and trytinol (10-15%), separating the aqueous layer containing 4-PAS, extracting the aqueous layer with methylene chloride 3-4 times, distilling the methylene chloride layer to obtain the semi to semi solid 4-PAS
e) adding toluene to solid to semi solid mass of 4-PAS, heating the mixture to 50-60° C. till residue is dissolved, adding acetic acid and refluxing the reaction mixture for 5 to 6 hrs., cooling the reaction mixture to 20-30° C. to obtain solid, separating the solid and washing with toluene, air drying at 35-40° C. till constant weight to obtain 6-PAS;
f) adding thionyl chloride to a mixture of suspension of 6-PAS and triphenylphosphine oxide in toluene while maintaining the temperature at 10-15° C., refluxing the reaction mixture for 2.5 to 3 hrs., cooling the reaction mixture to 10-15° C., adding a methanol water mixture (60-65% methanol in water), stirring further at 10-15° C. for 1 hr. to obtain a solid mass, separating the solid mass, washing the semi sold mass initially with a mixture of methanol water (60-65% methanol in water) and finally with water till pH of filtrate is 6.0, drying the solid in air at 35-40° C. till constant weight to obtain TOSPA of formula (5);
g) adding a solution of tetraalkylammonium hydroxide in methanol to a suspension of TOSPA in methanol to obtain the reaction mixture of pH 9, stirring the reaction mixture at 20-25° C. for 2 hrs neutralizing the reaction mixture with methanolic HCL, distilling methanol under vacuum to obtain crude sucralose, dissolving the crude sucralose in water and extracting with Methyl iso butyl ketone (MIBK), separating MIBK layer, treating MIBK layer with charcoal, precipitating the pure sucralose by partial distillation of MIBK under vacuum.

In still another embodiment the yield of 6-PAS using chloroform is about 55% and using toluene with aqueous process is about 70%

In a feature of the present invention the residue containing tritinol after extracting 4-PAS can be converted back to trityl chloride, which can be reused for tritylation.

In yet another feature of the invention, toluene layer after the bi-carbonate extraction of 4-PAS leaves behind a mixture of 85% trityl chloride and 15% trytinol. The reaction mass is converted into trityl chloride of purity minimum of 97% which can be reused for the preparation of TRISPA and tetra alkyl ammonium hydroxide is used for de-acetylation of TOSPA.

This reduces the cost as trityl chloride is a very costly reagent and hence the present process provides economic advantages over the prior art processes.

To illustrate the working of the present invention we are citing the following examples. These examples are just indicative and should not be considered to limiting to these examples. The applications would vary according to the specific characteristics of the chemicals and thus this example cannot be construed to be exhaustive.

### EXAMPLES

### Tritylation and Acetylation

### Preparation of 6, 1′,6′-TRI-O-TRITYL-SUCROSE PENTA ACETATE (TRISPA)

In a perfectly dry 1 litre 4 neck RB flask fitted with mechanical stirrer, thermometer pocket, solid addition funnel and condensor is placed Sucrose (25gm,0.073 mole) mixed with Triethylamine (29.04 gm, 0.287 mole), N,N-Dimethyl formamide (50 ml) and Dimethylaminopyridine (0.020 gm, 0.00016 mole) Trityl chloride (70.2 gm, 0.252 mole) is added in three portions within 1 to 1.5 hrs and stirred at 30-35° C. for around 16 hrs. The reaction mixture is then distilled under high vacuum (1-5 mm) to remove Dimethyl formamide not exceeding the temperature above 90° C. To the residual mass Acetic anhydride (52.92 gm, 0.518 mole) is added followed by anhydrous Sodium acetate (6.2 gm, 0.075 moles).After initial exotherm the reaction is maintained at 115 to 118° C. for three hrs. After cooling the reaction mass to 50° C. Methanol (200 ml) is added slowly to precipitate the product. It is stirred at 25 to 30° C. for 1-2 hrs and the precipitated solid is filtered, wash initially with Methanol followed by water to remove inorganic salts.

The product can be dried at 70-75° C. in a tray drier till its moisture content is less than 0.5%.

Yield is 50 to 53 gms (% Yield=53.5 to 56 Purity by HPLC=93 to 95%) [∝]_{D}25° C.=+66° to 67° (2.45% in Chloroform)]

### Example

### Detritylation and Acetylmigration

### Preparation of 2,3,4,3′4′-penta-O acetyl Sucrose (4 PAS) (not isolated) and 2,3,6,3′4′-penta-O acetyl Sucrose (6PAS)

In a perfectly dry 1 litre 4 neck RB flask fitted with mechanical stirrer, thermometer pocket, gas purging tube and condensor is placed dried 6,1′,6′-tri-O-trityl-sucrose penta acetate (Stage 1) (50 gm 0.039 mole) dissolved in 150 ml chloroform. The solution is cooled to −1 to −15° and Hydrochloric acid gas is passed through the solution within 10 mins to 1 hr. Once the reaction mixture shows the absence of starting material on TLC it is poured on to Sodium Bi-carbonate solution (12 gm in 120 ml water)and stirred at 20° C. for 0.5 hrs to 1.0 hrs. The reaction mass was allowed to settle. The chloroform layer is separated and the aqueous layer is washed with chloroform (2×50 ml).The total chloroform layer is mixed together and the solvent is distilled out initially at atmospheric pressure and finally by applying water vacuum. Then 100 ml water is added to the semisolid separated and heat to 85 to 95° C.

The solid is then filtered and this solid is again washed with 100 ml water and is filtered hot. The total water extract is cooled to 30° C. and saturated with about 50-55 gm Sodium Chloride. It is then extracted with Methylene chloride (2×100 ml).The Methylene chloride layer is dried over Sodium sulphate and the solvent is removed by distillation initially at atmospheric pressure and finally under vacuum. To the pale yellow solid to semi solid formed in the flask is added Toluene (75 ml) followed by Acetic acid (1.5 ml).

The reaction mixture is refluxed for 6 hrs. It is then cooled to 15 to 20° C. The solid seperated is filtered and washed with Toluene (10 to 15 ml). It is then air dried at 35 to 40° C. in a tray drier till constant weight. Yield=12 gms

[% Yield=55.6) [∝]_{D}25° C.=+30° to +33° (3.1% in Chloroform)]

### Example

### Chlorination

### Preparation of 4, 1′, 6′-TRICHLORO-4, 1′6-TRIDEOXYGALACTOSUCROSE PENTAACETATE (TOSPA)

In a perfectly dry 1 litre 4 neck RB flask fitted with mechanical stirrer, thermometer pocket and condensor is placed 6 PAS (15.5 gm , 0.028 mole) in Toluene 45 ml. Add Triphenylphosphine oxide (7.8 gms, 0.028 mole) to it. The above suspension is cooled to 10° C. and Thionyl chloride (14 gm, 0.118 mole) is added dropwise maintaining the temperature between 10-15° C. Stir at 25-30° C. for 0.5 hrs and then reflux the reaciton mixture for 2.5 to 3.0 hrs at 110° C. .Cool the reaction mixture to 10° C. and add 60% aqueous Methanol (40 ml).Stir it for 1-2 hrs at 10-15° C. .The solid separated is filtered and wash with water till the pH of water is neutral. The material is slurried with 12 ml Toluene at 50-60° C. for 1 hr, cool to 10-15° C. and filter to get product. Yield =11.4 gm

(% Yield =66.86) [∝]_{D}25° C.=+65.5° to 68.5° (0.9% in Chloroform)]

### Example 5

### Deacetylation

### Preparation of 1,6-DICHLORO-1,6-DIDEOXY-β-D-FRUCTOFURANOSYL-4-CHLORO-4-DEOXY-α-D-GALACTOPYRANOSIDE.(SUCRALOSE)

In a dry 1 litre 4 neck RB flask fitted with mechanical stirrer ,thermometer pocket and condensor is placed TOSPA (15 gm, 0.0247 mole) in Methanol (60 ml), cool to 20° C. and 2.5% solution of Tetra butylammonium hydroxide in Methanol is added to it till the pH of the reaction mixture is 9. Then stir at 20° C. for 2.5 to 3 hrs till TLC shows the complete formation of Sucralose.Then neutralise the reaction with Methanolic HCL

(pH 6-6.5). Then distil off Methanol completely under high vacuum (1-5mm) at a temperature below 50° C. .Then cool to 25° C. and add 15 ml of water to get a clear solution, extract with Methyl Isobutyl Ketone (MIBK) (2×15 ml & 1×10 ml) Collect all organic layer, add Charcoal (0.1 gm) stir for 0.5 hrs at 25-30° C. and filter. MIBK from the filterate is distilled under high vacuum till the solution in the flask becomes turbid. Then cool the reaction flask to 5° C. and maintain for 1 hr. The solid separated is filtered, wash with chilled MIBK (5 ml) and dry at 35-40° C. under high vacuum (1-5 mm).

Yield=6.75 gm (% Yield=68.8)

Detailed descriptions of the preferred embodiment are provided herein; however, it is to be understood that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or matter.

1. A process for the preparation of sucralose of formula (1) 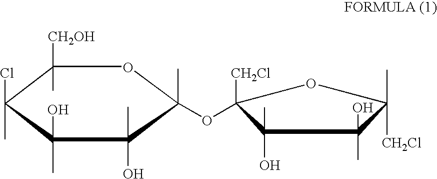
comprising the steps of
a) preparing 6,1,6″-tri-o-triyl sucrose pentaacetate (TRISPA) of formula (2) from sucrose using triethyl amine or pyridine 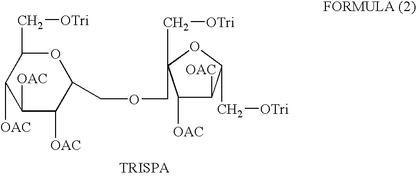
b) preparing 2,3,6,3′,4′-Penta-o-acetylsucrose (6-PAS) of formula (4) using TRISPA of formula (2) via in situ preparation of a compound 4-PAS of formula (3) 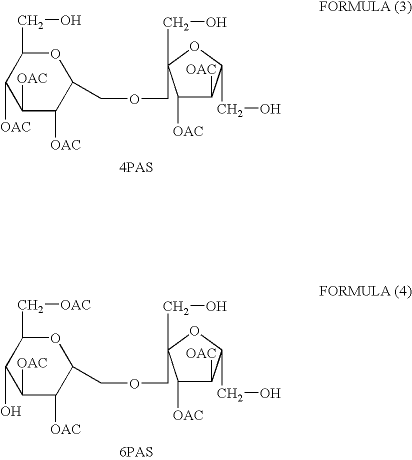
c) subjecting 6-PAS to chlorination to obtain compound TOSPA of formula (5) by using aqueous methanol 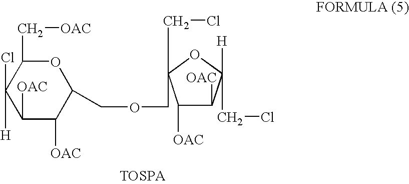
d) deacetylating TOSPA for formula (5) to sucralose of formula (1) using quaternary ammonium hydroxide.

2. process as claimed in claim 1, which comprises the steps of:
a) preparing a mixture of sucrose, triethyl amine and a catalyst in N-N-dimethyl formamide (DMF) at ambient temperature, adding excess trityl chloride successively in three equal portions in 1 to 1.5 hours, continuing stirring at ambient temperature for about 16 hours, distilling DMF under vacuum below 85° C., to obtain a thick paste of crude tritrytil sucrose, dissolving the thick paste in acetic anhydride at a temperature in the range of 50-60° C., adding anhydrous sodium acetate while maintaining the reaction temperature at 110 to 115° C. for 3 to 4 hours, cooling the reaction mixture to 45-50° C., precipitating a solid by adding methanol, separating and washing the solid successively with methanol and water to remove inorganic salts, drying the solid at 65-75° C. till constant weight to obtain TRISPA;
b) dissolving dry TRISPA in chloroform to form a solution, cooling the solution to a minimum of −1° C. to −15° C., passing dry HCL gas through the solution for a maximum of 15 to 60 minutes, to obtain a reaction mixture having temperature of −5 to −3° C., quickly pouring the reaction mixture into aqueous sodium bicarbonate solution under constant stirring at pH 7.5 to 8 and separating the chloroform layer containing 4-PAS and trytinol, separating the aqueous layer, washing the aqueous layer 2 to 3 times with chloroform, removing the chloroform by distillation to obtain a thick pasty mass of a mixture containing 4-PAS and tritinol, extracting the thick pasty mass with hot water at 85-90° C., 3 to 4 times to obtain 4-PAS in aqueous solution, cooling the hot water extract to 25-30° C., saturating with sodium chloride, extracting the saturated solution with methylene chloride 3-4 times, distilling the methylene chloride layer to obtain the semi to semi solid 4-PAS;
c) adding toluene to solid to semi solid mass of4-PAS, heating the mixture to 50-60° C. until residue is dissolved, adding acetic acid and refluxing the reaction mixture for 5 to 6 hours, cooling the reaction mixture to 20-30° C. to obtain solid, separating the solid and washing with toluene, air drying at 35-40° C. until constant weight to obtain 6-PAS;
d) adding thionyl chloride to a mixture of suspension of 6-PAS and triphenylphosphine oxide in toluene while maintaining the temperature at 10-15° C., refluxing the reaction mixture for 2.5 to 3 hours, cooling the reaction mixture to 10-15° C., adding a methanol water mixture (60-65% methanol in water), stirring further at 10-15° C. for 1 hour to obtain a solid mass, separating the solid mass, washing the semi solid mass initially with a mixture of methanol water (60-65% methanol in water) and finally with water until pH of filtrate is 6.0, drying the solid in air at 35-40° C. until constant weight to Obtain TOSPA of formula (5); and
e) adding to a solution of tetraalkyl ammonium hydroxide in methanol to a suspension of TOSPA in methanol to obtain the reaction mixture of pH 9, stirring the reaction mixture at 20-25° C. for 2 hours neutralizing the reaction mixture with methanolic HCL, distilling methanol under vacuum to obtain crude sucralose, dissolving the crude sucralose in water and extracting with methyl iso butyl ketone (MIBK), separating MIBK layer, treating MIBK layer with charcoal, and precipitating the pure sucralose by partial distillation of MIBK under vacuum.

3. The process as claimed in claim 2, wherein said process comprises preparing a mixture of sucrose in pyridine, adding to this mixture excess trityl chloride successively in three equal portions in 1 hour intervals, heating the reaction mixture to 45 to 50° C. for a minimum of 5 hours distilling pyridine under vacuum at a temperature below 80-85° C. to obtain a residue, cooling the residue to 50° C., adding acetic anhydride to obtain a homogenous mass, stirring the homogenous mass at ambient temperature for 3 to 3.5 hours heating the homogenous mass to 100-105° C. for a minimum of two hours, precipitating by adding methanol, separating a solid and washing the solid successively with methanol and water to remove inorganic salts, and drying the solid at 65-75° C. until constant weight to obtain TRISPA.

4. The process as claimed in claim 2, wherein said process comprising dissolving wet or dry TRISPA in a toluene, removing water by conventional methods if wet TRISPA is used, cooling the clear solution to −1 to −15° C., passing dry HCL gas through the clear solution for a maximum of 15 to 60 minutes, to obtain a reaction mixture having temperature of −5 to −3° C., quickly adding aqueous sodium bicarbonate into the reaction mixture under constant stirring at pH 7.5 to 8 and separating the toluene layer containing mixture of trytil chloride(85-90%) and trytinol (10-15%), separating the aqueous layer containing 4-PAS, extracting the aqueous layer with methylene chloride 3-4 times, distilling the methylene chloride layer to obtain the semi to semi solid 4-PAS.

5. The process as claimed in claim 3, wherein ambient temperature is 75 to 80° C.

6. The process as claimed in claim 2, wherein said residue containing said trytinol after extracting 4-PAS is converted back to trityl chloride.

7. The process as claimed in claim 4, wherein said toluene layer containing trityl chloride 85 to 90% and trytinol 10 to 15% is converted back to trityl chloride which is minimum of 97 percent.

8. A The process as claimed in claim 6, wherein said trityl chloride is reused for tritylation.

9. The process in claim 1, wherein tetra alkyl ammonium hydroxide is used for de-acetylation of TOSPA.

10. The process as claimed in claim 1, wherein methanolic hydrochloric acid is used for the neutralization after de-acetylation.
